(19) United States
(12) Patent Application Publication
(10) Pub. No.: US 2008/0221336 A1
(43) Pub. Date: Sep. 11, 2008

(54) Pparalpha/Gamma Agonists and Processes of Preparing

(76) Inventors: Christine E. Garrett, Bridgewater, NJ (US); Guang-Pei Chen, Livingston, NJ (US); George Tien-San Lee, Towaco, NJ (US); Xinglong Jiang, Hillsborough, NJ (US); Michael J. Girgis, Montville, NJ (US); James Anthony Vivelo, Basking Ridge, NJ (US); Beata Sweryda-Krawiec, Whitehouse Station, NJ (US); Dimitris Papoutsakis, Acton, MA (US); Piotr Karpinski, Lincoln Park, NJ (US); Prasad Koteswara Kapa, Parsippany, NJ (US); Ada Skorodinsky, Livingston, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/995,261

(22) PCT Filed: Jul. 12, 2006
(86) PCT No.: PCT/US06/27441

§ 371 Date: Jan. 10, 2008

Related U.S. Application Data
(60) Provisional application No. 60/698,801, filed on Jul. 13, 2005.

(51) Int. Cl. C07D 263/32

(52) U.S. Cl. 548/235

(57) ABSTRACT

This invention provides a method for preparing compounds having the structure [Formula (I)] wherein L is [Formula (A)] or [Formula (B)] in which R₁ is hydrogen or optionally substituted alkyl, n is zero or 1 and m is 1; wherein R is H, halogen, optionally substituted C₁₋₆alkyl or C₁₋₆alkoxy; wherein Z is a bond, O or S; wherein p is an integer from 1 to 5; wherein Q is a bond provided that Z is not a bond when p is 1; or Q is O, S or —C(O)NR₆— in which R₆ is hydrogen, optionally substituted alkyl or cycloalkyl; or Q is —NR₆—, —NR₅C(O)NH— or —NR₅C(O)O— in which R₅ is hydrogen, alkyl or aralkyl provided that p is not 1; wherein W is cycloalkyl, aryl or heterocyclyl; or W and R₆ form a 9- to 10-membered bicyclic ring, which may be optionally substituted or may contain oxygen, nitrogen or sulfur.

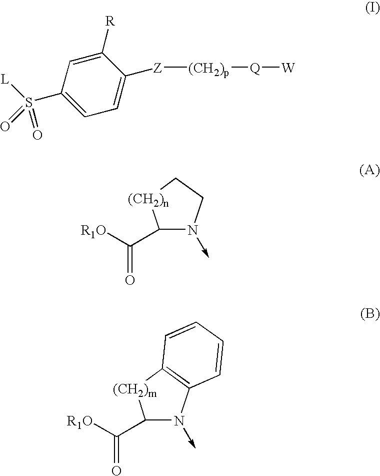

Throughout this application, various publications are referenced. The disclosures of these publications in their entireties are hereby incorporated by reference into this application in order to more fully describe the state of the art known to those skilled therein as of the date of this invention.

The present invention relates to compounds which are Peroxisome Proliferators-Activated Receptor (PPAR) α and γ agonists and new processes of preparing these compounds. More specifically, this invention relates to a new process for preparing (2R)-2,3-Dihydro-1-[[4-[[5-methyl-2-[4-(trifluoromethyl)phenyl]-4-oxazolyl]methoxy]phenyl]sulfonyl]-1H-indole-2-carboxylic acid, or potassium hydrogen bis((2R),-2,3-dihydro-1-[[4-[[5-methyl-2-[4-(trifluoromethyl)phenyl]-4-oxazolyl]methoxy]phenyl]sulfonyl]-1H-indole-2-carboxylate), or analogous pharmaceutically acceptable salts thereof.

The compounds of the invention are potent agonists of Peroxisome Proliferator-Activated Receptors (PPARs). Accordingly, the compounds of the invention are useful for the treatment of conditions mediated by the PPAR receptor activity in mammals. Such conditions include dyslipidemia, hyperlipidemia, hypercholesteremia, atherosclerosis, hypertriglyceridemia, heart failure, myocardial infarction, vascular diseases, cardiovascular diseases, hypertension, obesity, Syndrome-X, inflammation, arthritis, cancer, Alzheimer's disease, skin disorders, respiratory diseases, ophthalmic disorders, inflammatory bowel diseases, ulcerative colitis, type-1 and type-2 diabetes, Crohn's disease, stroke, intermittent claudication, restenosis after PCTA, obesity including reduction in CV risk in obese patients, hypofibrinolysis, hypercoaguable state, metabolic/cardiometabolic syndrome, elevated CRP, appearance of microalbuminuria, reduction of proteinuria, renal failure (DM, non-DM), NASH (non alcoholic steato hepatitis) non-alcoholic fatty liver, CV events in patients with high CRP, vascular dementia, psoriasis, ischaemia reperfusion injury, asthma, COPD, eosinophilia, RA, airway hyperresponsiveness (AHR), inflammatory digestive diseases (e.g. ulcerative colitis) diseases of antigen-induced inflammatory responses, impaired glucose tolerance, hyperglycemia, insulin resistance, and for the improvement of cardiac metabolism and cardioprotection in heart transplant patients. In addition, the compounds of the invention are also useful in the facilitation of smoking cessation, temporary abstinence or smoking reduction and therefore prevention, delay of progression or treatment of conditions associated with smoking such as craving for nicotine and the increased appetite, dysphoria or depressed mood, sleeplessness, irritability, frustration, anger, anxiety, difficulty in concentrating and restlessness.

Dosages and routes of administration suitable for the compounds of the instant invention are well known in the art and can be found, inter alia, in PCT International Publication Number WO 03/043985 A1, published on May 30, 2003, the contents of which are hereby incorporated by reference into this application.

Surprisingly, it was found that these compounds could be obtained in high purity using a simplified three-step method wherein the first intermediate is isolated by filtration directly from the reaction mixture, the second intermediate is isolated by filtration after quenching the reaction mixture with water, and the product is isolated either in the free acid and the desired crystalline form using PVP, i.e. (2R)-2,3-Dihydro-1-[[4-[[5-methyl-2-[4-(trifluoromethyl)phenyl]-4-oxazolyl]methoxy]phenyl]sulfonyl]-11H-indole-2-carboxylic acid, or as a potassium, hydrogen bis carboxylate form, i.e. potassium hydrogen bis((2R),-2,3-dihydro-1-[[4-[[5-methyl-2-[4-(trifluoromethyl)phenyl]-4-oxazolyl]methoxy]phenyl]sulfonyl]-1H-indole-2-carboxylate).

This invention provides a method of preparing a compound having the structure

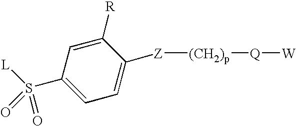

wherein L is

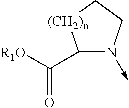

radical in which R₁ is hydrogen or optionally substituted alkyl, and n is zero or 1; or
   L is

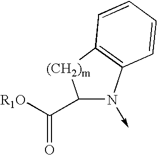

radical in which R₁ is hydrogen or optionally substituted alkyl, and m is 1;
wherein R is H, halogen, optionally substituted C₁₋₆alkyl or C₁₋₆alkoxy;
wherein Z is a bond, O or S;
wherein p is an integer from 1 to 5;
wherein Q is a bond provided that Z is not a bond when p is 1; or
   Q is O, S or —C(O)NR₆— in which R₆ is hydrogen, optionally substituted alkyl or cycloalkyl; or
   Q is —NR₆—, —NR₅C(O)NH— or —NR₅C(O)O— in which R₅ is hydrogen, alkyl or aralkyl provided that p is not 1;
wherein W is cycloalkyl, aryl or heterocyclyl; or
   W and R₆ taken together with the nitrogen atom to which they are attached form a 9- to 10-membered bicyclic ring, which may be optionally substituted or may contain another heteroatom selected from oxygen, nitrogen and sulfur;
the method comprising the steps of
(a) reacting Cl—(CH₂)ₚ-Q-W in the presence of a solvent, and

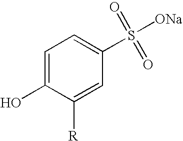

to provide

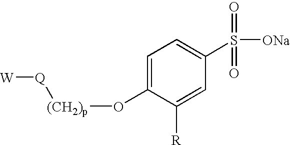

(b) treating the product of step (a) with dimethylformamide (DMF), acetonitrile, and thionyl chloride to provide

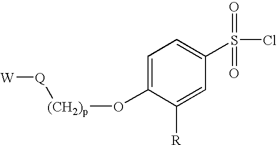

(c) reacting the product of step (b) with L in the presence of NaOH to provide the compound having the structure

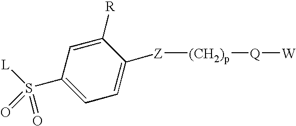

or a pharmaceutically acceptable salt thereof.

This invention also provides the above method,
wherein L is

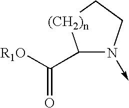

radical in which R₁ is hydrogen or optionally substituted alkyl, and n is zero or 1,
   resulting in product of step (c) shown below.

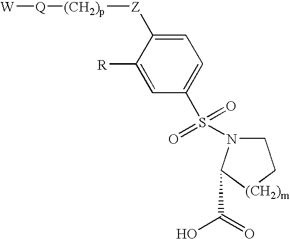

This invention also provides the above method,
wherein L is

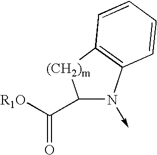

radical in which R₁ is hydrogen or optionally substituted alkyl, and m is 1,
   resulting in the product of step (c) shown below.

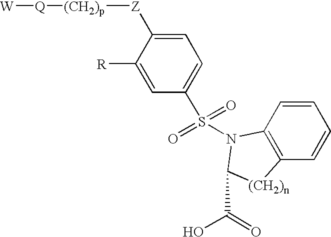

This invention also provides the above method further comprising a step (d) of treating the product of step (c) with KOH to provide the product having the structure

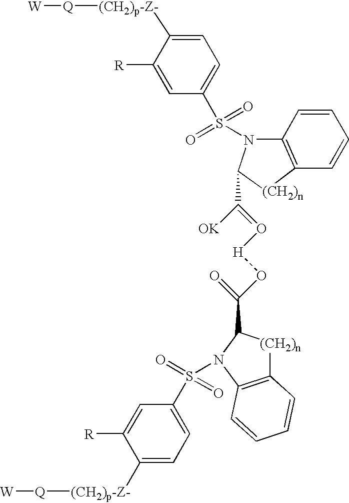

This invention further provides a step (e) of recrystallization of the product of step (d) in the presence of 2-propanol and water, or other suitable organic solvents which are miscible with water.

The product of step (d) may also be represented as

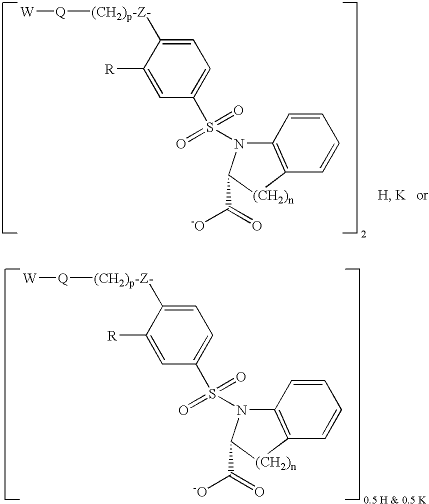

This invention further provides the above method further comprising a step (f) of milling the recrystallized product of step (e).

This invention also provides the above method, wherein R is a hydrogen, Z is O, p is 1, Q is a bond, and W is

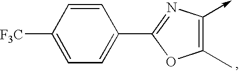

providing the product of step (c) having the structure

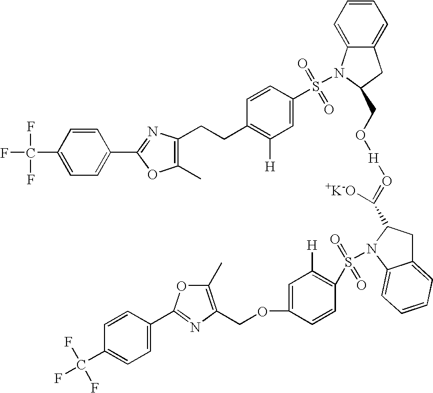

The above structure can also be represented as

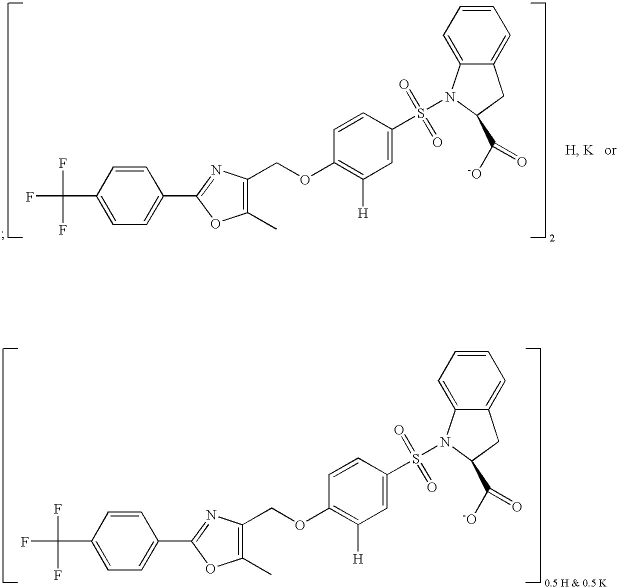

or an analogous pharmaceutically acceptable salt thereof.

In the practice of this invention, analogous pharmaceutically acceptable salts include alkaline earth metal salts such as a calcium salt or a magnesium salt. Other pharmaceutically acceptable salts may be, but are not limited to, mesylate, maleate, fumarate, tartrate, hydrochloride, hydrobromide, esylate, p-toluenesulfonate, benzoate, acetate, phosphate and sulfate salts.

This invention further provides the above method further comprising a step (e) of recrystallizing the product of step (d). The recrystallizing may be in the presence a suitable water miscible solvent, for example, alcohol or acetonitrile. For example, the recrystallizing may be in the presence of 2-propanol and water.

This invention further provides the above method further comprising a step (f) of milling the recrystallized product of step (e).

The solvent of step (a) may be 1-methyl-2-pyrrolidinone (NMP) or other suitable solvents such as, but not limited to, dimethylformamide (DMF) or dimethylacetamide (DMA).

This invention provides the above method, wherein the product of step (b) was isolated by water quenching.

This invention provides the above method, wherein the product of step (c) was isolated by adding water and adjusting the pH to 1-2.

This invention also provides the above method, wherein R is H, halogen, optionally substituted C₁₋₆alkyl or C₁₋₆alkoxy; Z is O or S; p is 2; W is aryl or heterocyclyl; and Q is a —NR₆— in which R₆ is lower alkyl. Specifically, R may be H, chloro, n-propyl, or methoxy.

This invention also provides the above method, wherein R is H, halogen, optionally substituted C₁₋₆alkyl or C₁₋₆alkoxy; Z is a bond; p is 2; W is aryl or heterocyclyl, or W and R₆ taken together with the nitrogen atom to which they are attached form a 9- to 10-membered bicyclic ring, which may be optionally substituted or may contain another heteroatom selected from oxygen, nitrogen and sulfur; and Q is a —C(O)NR₆— in which R₆ is optionally substituted alkyl. Specifically R may be H, chloro, n-propyl, or methoxy.

This invention also provides the above method, wherein R is H, halogen, optionally substituted C₁₋₆alkyl or C₁₋₆alkoxy; Z is a bond, O, or S; p is 2 or 3; W is aryl or heterocyclyl; and Q is O or S. Specifically, R may be H, chloro, n-propyl, or methoxy.

This invention also provides the above method, wherein R is H, halogen, optionally substituted C₁₋₆alkyl or C₁₋₆alkoxy; Z is a bond, O, or S; p is 2 or 3; W is selected from the group consisting of:

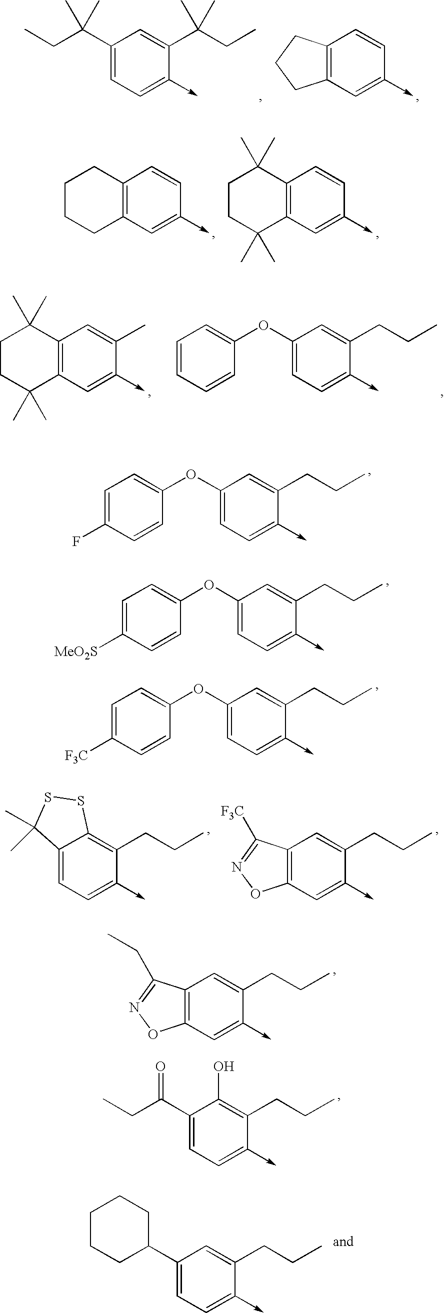

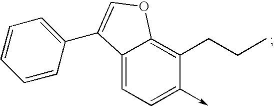

### and Q is O or S.

This invention also provides the above method, wherein R is H, halogen, optionally substituted C₁₋₆alkyl or C₁₋₆alkoxy; z is O or S; p is 1 or 2; W is aryl or heterocyclyl; and Q is a bond. Specifically, R may be H, chloro, n-propyl, or methoxy.

This invention also provides the above method, wherein R is H, halogen, optionally substituted C₁₋₆alkyl or C₁₋₆alkoxy; Z is O or S; p is 1 or 2; W is selected from the group consisting of:

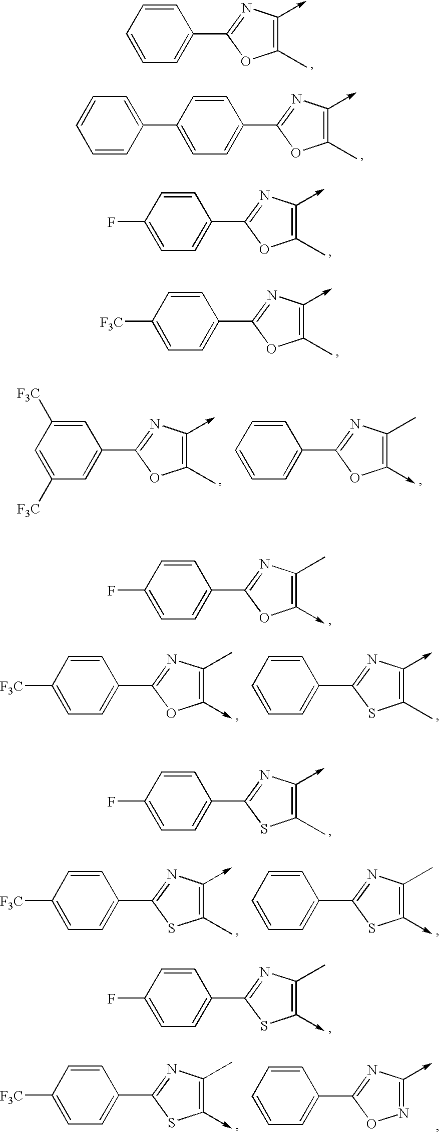

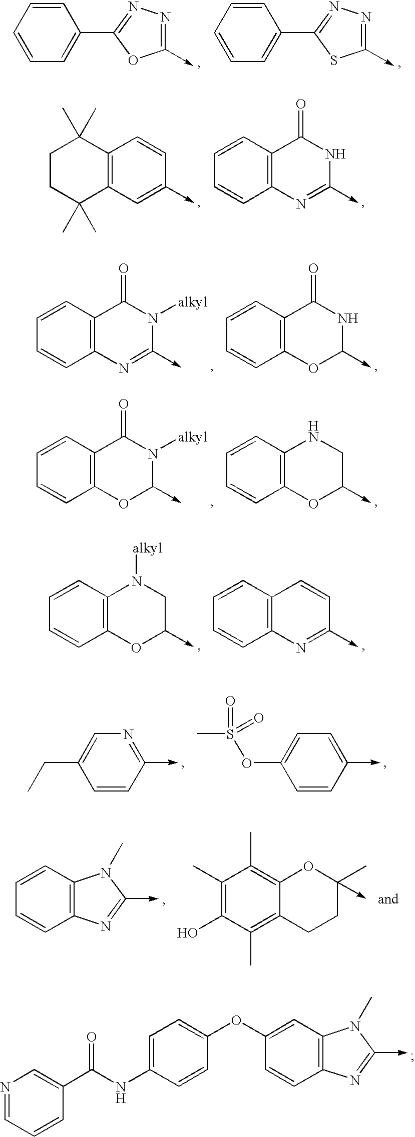

and Q is a bond.

This invention also provides the above method, wherein R is H, halogen, optionally substituted C₁₋₆alkyl or C₁₋₆alkoxy; Z is O or S; p is 2; W is selected from the group consisting of:

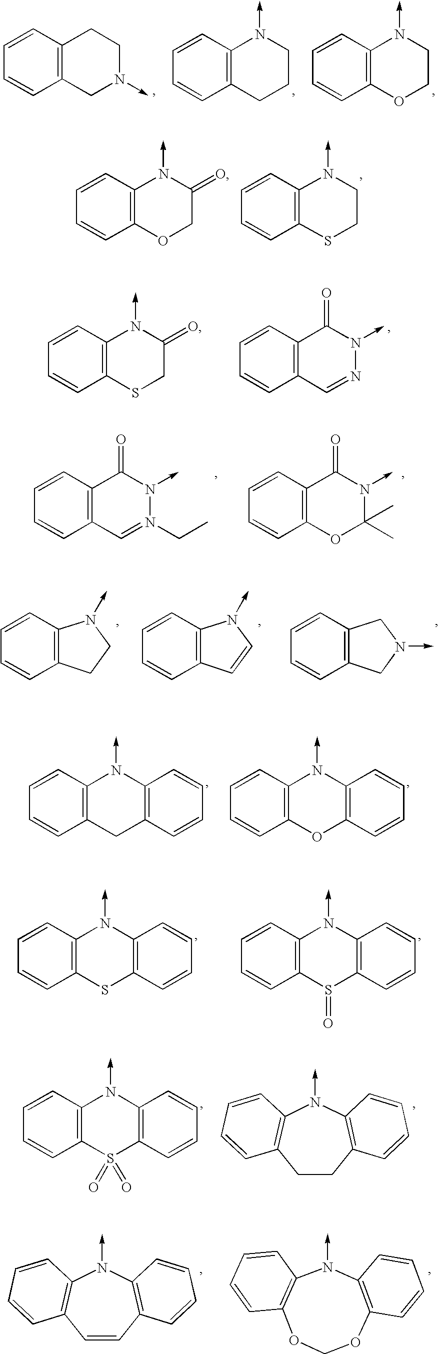

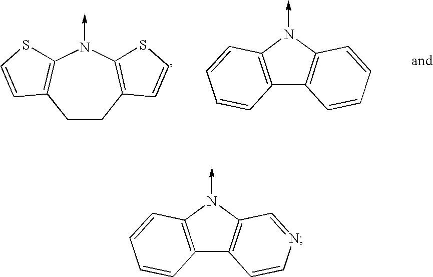

and Q is a bond.

In one embodiment, this invention further provides a compound having the structure

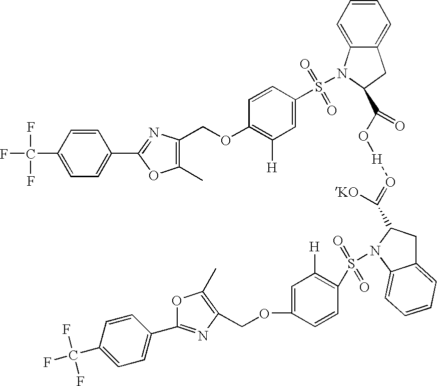

Alternatively, this invention provides a compound having the structure

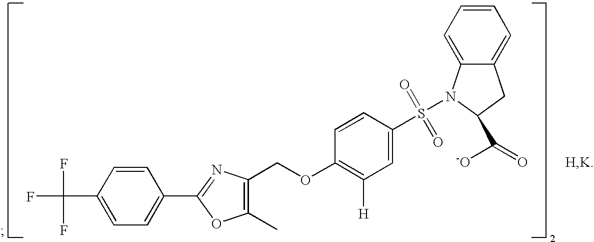

Alternatively, this invention provides a compound having the structure

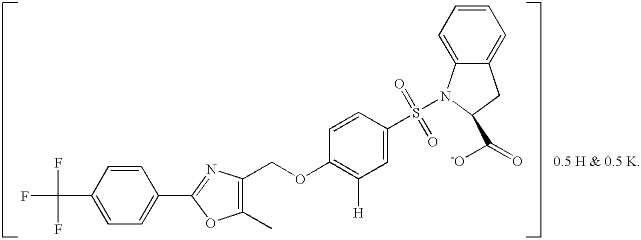

In particular, this invention provides a method for preparing a compound having the structure

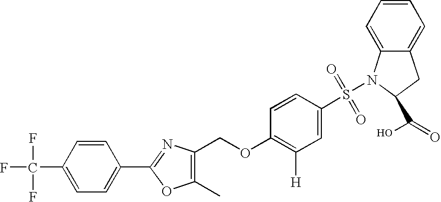

and a hemi potassium salt having the structure

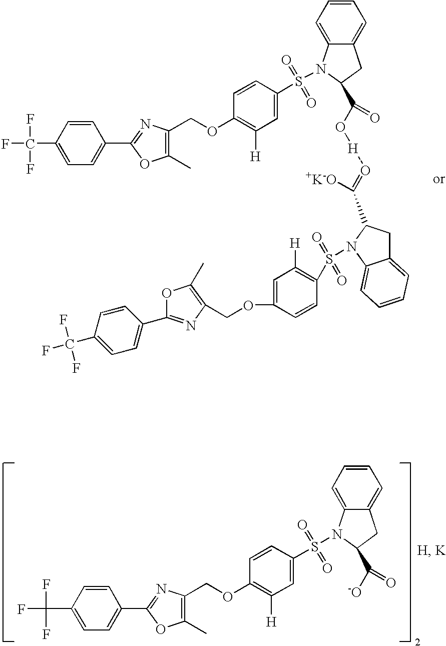

which comprises the reaction steps outlined in Scheme 1 below.

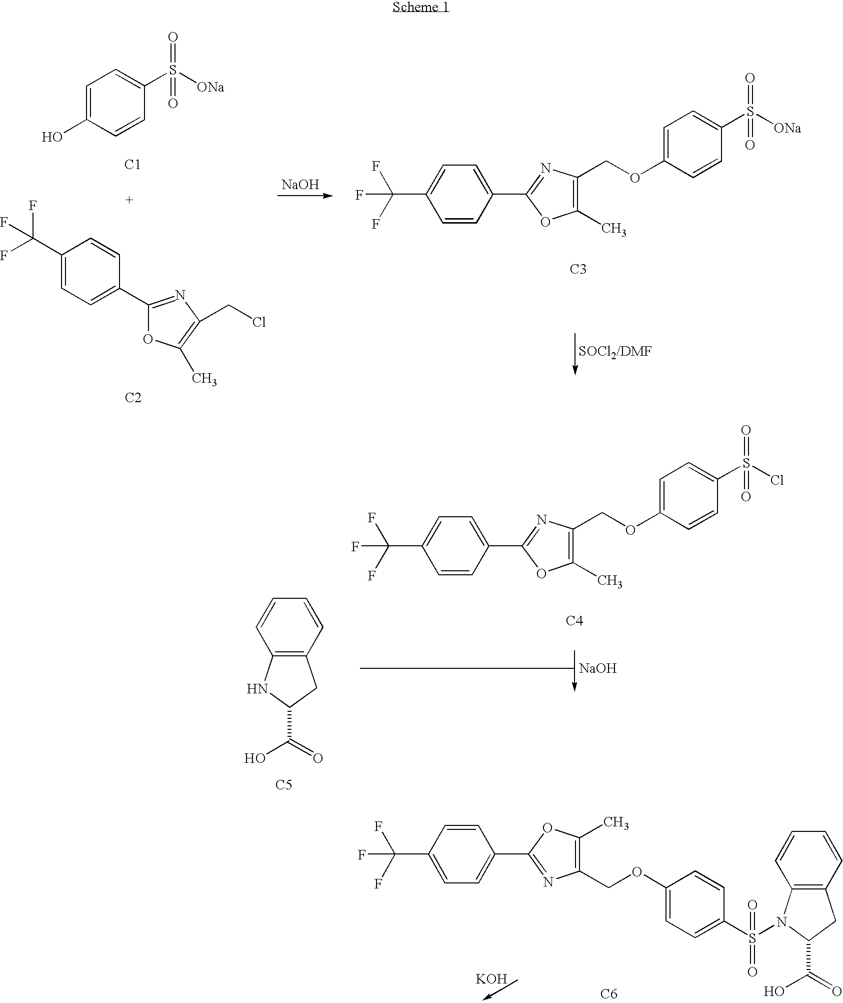

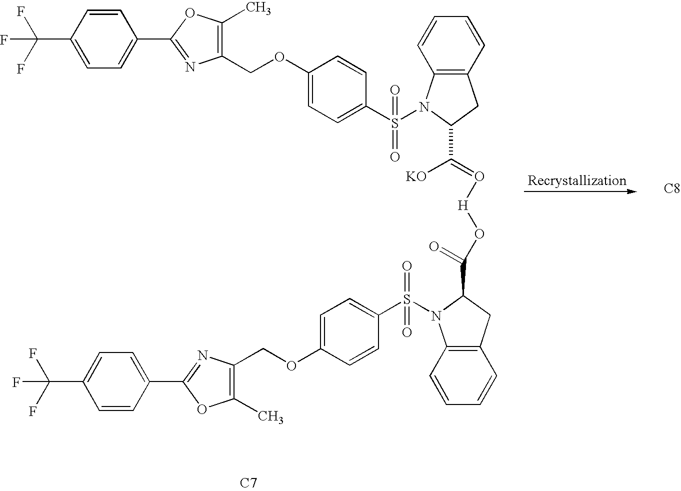

Listed below are definitions of various terms used to describe the compounds of the instant invention. These definitions apply to the terms as they are used throughout the specification and the claims unless they are otherwise limited in specific instances either individually or as part of a larger group.

The term “optionally substituted alkyl” refers to unsubstituted or substituted straight or branched chain hydrocarbon groups having 1 to 20 carbon atoms, preferably 1 to 7 carbon atoms.

Exemplary unsubstituted alkyl groups include methyl, ethyl, propyl, isopropyl, n-butyl, f-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halo, hydroxy, cycloalkyl, alkanoyl, alkoxy, alkyloxyalkoxy, alkanoyloxy, amino, alkylamino, dialkylamino, alkanoylamino, thiol, alkylthio, alkylthiono, alkylsulfonyl, arylsulfonyl, heteroarylsulfonyl, sulfonamido, nitro, cyano, carboxy, alkoxycarbonyl, aryl, alkenyl, alkynyl, aralkoxy, guanidino, heterocyclyl including indolyl, imidazolyl, furyl, thienyl, thiazolyl, pyrrolidyl, pyridyl, pyrimidyl, piperidyl, morpholinyl and the like.

The term “halogen” or “halo” refers to fluorine, chlorine, bromine and iodine.

The term “cycloalkyl” refers to optionally substituted monocyclic, bicyclic or tricyclic hydrocarbon groups of 3 to 12 carbon atoms, each of which may optionally be substituted by one or more substituents, such as alkyl, halo, oxo, hydroxy, alkoxy, alkanoyl, amino, alkylamino, dialkylamino, thiol, alkylthio, nitro, cyano, carboxy, carboxyalkyl, alkoxycarbonyl, alkyl- and arylsulfonyl, sulfonamido, heterocyclyl and the like.

Exemplary monocyclic hydrocarbon groups include, but are not limited to, cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicycIo[3.1.1]heptyl, 2,6,6-trimethylbicyclo[3.1.1]heptyl, bicyclo[2.2.2]octyl and the like.

Exemplary tricyclic hydrocarbon groups include adamantyl and the like. The term “alkoxy” refers to alkyl-O—.

The term “aryl” refers to monocyclic or bicyclic aromatic hydrocarbon groups having 6 to 12 carbon atoms in the ring portion, such as phenyl, naphthyl, tetrahydronaphthyl, biphenyl and diphenyl groups, each of which may optionally be substituted by one to four substituents, such as alkyl, halo, hydroxy, alkoxy, alkanoyl, alkanoyloxy, optionally substituted amino, thiol, alkylthio, nitro, cyano, carboxy, carboxyalkyl, alkoxycarbonyl, alkylthiono, alkyl- and arylsulfonyl, sulfonamido, heterocycloyl and the like.

The term “aralkyl” refers to an aryl group bonded directly through an alkyl group, such as benzyl.

The term “heterocyclyl” or “heterocyclo” refers to an optionally substituted, fully saturated or unsaturated, aromatic or non-aromatic cyclic group, for example, which is a 4- to 7-membered monocyclic, 7- to 12-membered bicyclic, or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, dihydroquinazolinyl (such as 3,4-dihydro-4-oxo-quinazolinyl), phthalazinyl and the like. Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” includes substituted heterocyclic groups. Substituted heterocyclic groups refer to heterocyclic groups substituted with 1, 2 or 3 of the following: alkyl; hydroxy (or protected hydroxy); halo; oxo (i.e. ═O); optionally substituted amino, alkylamino or dialkylamino; alkoxy; cycloalkyl; carboxy; heterocyclooxy; alkoxycarbonyl, such as unsubstituted lower alkoxycarbonyl; mercapto; nitro; cyano; sulfonamido, sulfonamidoalkyl, sulfonamidoaryl or sulfonamidodialkyl; aryl; alkylcarbonyloxy; arylcarbonyloxy; arylthio; aryloxy; alkylthio; formyl; carbamoyl; aralkyl; or aryl substituted with alkyl, cycloalkyl, alkoxy, hydroxy, amino, alkylamino, dialkylamino or halo.

To fully understand the present invention by way of example, below is provided a more detailed step by step analysis of Scheme 1.

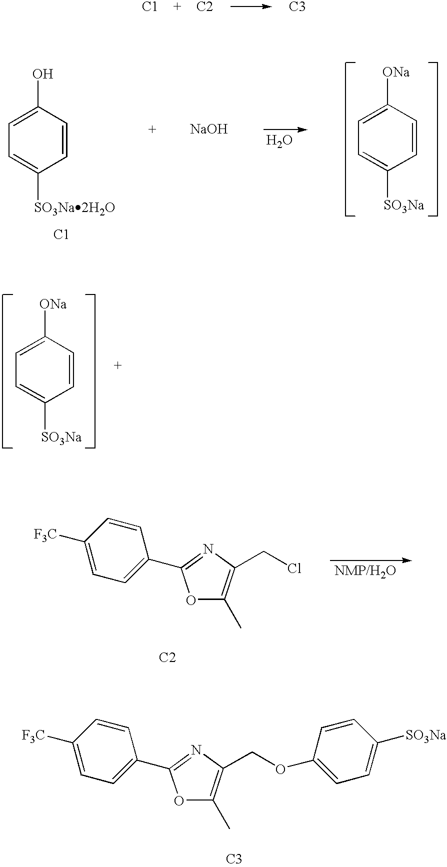

### Procedure:

Charge a 2-L reactor with 39.4 g of 50% sodium hydroxide and 324 g (324 ml) of water. Stir and to the clear, colorless solution, add 110.4 g (0.472 mol) of C1. Continue stirring for 15 minutes. Slowly add 517 g (500 mL) of 1-methyl-2-pyrrolidinone (NMP) and adjust the addition rate while maintaining the batch temperature below 30° C. The reaction mixture changes from clear solution to misty to cloudy then to thin white slurry. Stir at the temperature for 30 min. Add 120.0 g (0.435 mol) of C2. Raise the batch temperature to 60° C. The reaction mixture changes to thick white slurry. Hold for 6 h. Cool the reaction mixture to 50° C., add 560 g (560 mL) of water to the slurry and stir at 50° C. for 30 min. Cool the reaction mixture to 22° C. and stir the slurry at 22° C. for 1 h. Filter the mixture. Use 393 g (500 mL) of acetonitrile to wash and rinse the reactor, and wash the filter-cake. Wash the filter cake with 500 g (500) mL of water. Dry the wet cake at 65° C. under house vacuum (˜25 mbar) at least for 18 h to give 161.5 g of C3 (dry) as a white solid.

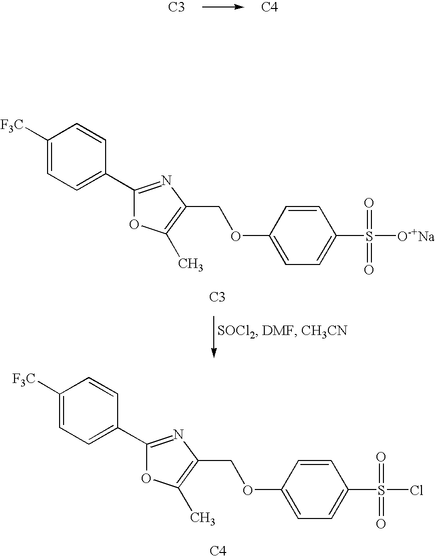

### Procedure:

A 2-L reactor was charged with 128.8 g of [4-[[5-methyl-2-[4-(trifluoromethyl)phenyl]-4-oxazolyl]methoxy]-phenyl]sulfonic acid, sodium salt (C3), 5.6 g of N,N-dimethylformamide, and 484 g of acetonitrile. Warm the reaction mixture to 40° C. over 15 min to give a white suspension. Add 85 g of thionyl chloride slowly into pot at 40° C. over 30 min, then heat the contents to 45° C. over 10 min. Maintain the suspension at 45° C. under an efficient stirring for 2 h. Add 1 kg of water into pot at 15° C. over 30 min to obtain a pale yellow suspension, then stir the contents at 20° C. for 1.5 h. Filter the solids through a polypropylene filter cloth in a Büchner funnel then wash the flask and filter cake once with 616 g of water. Dry the wet cake at 60° C. (30 mbar) for 18 h to give 122.7 g of [4-[[5-methyl-2-[4-(trifluoromethyl)phenyl]-4-oxazolyl]methoxy]phenyl]-sulfonyl chloride (C4) as a pale yellow solid. mp 90-93° C.

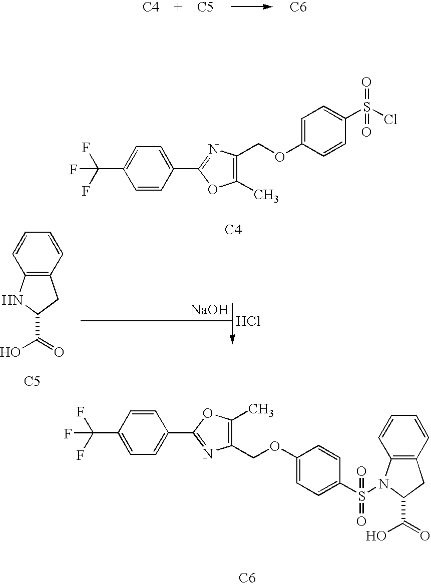

### Procedure:

A 500 mL 4-necked flask was charged with 9.75 g of (R)-indoline-2-carboxylic acid (C5), 60 g of water, and 5.3 g of 50% sodium hydroxide aq. solution. Stir the reaction mixture to 20° C. for 15 min. Add 118 g of acetonitrile into pot at 20→15° C., and stir at 15° C. for 15 min to give a light tan homogenous solution. Add 21.6 g of [4-[[5-methyl-2-[4-(trifluoromethyl)phenyl]-4-oxazolyl]methoxy]phenyl]sulfonyl chloride (C4) into pot at 15-18° C. in 3 portions over 30 min. Maintain the contents at 18° C. under an efficient stirring for 1.5 h and stir at 20° C. for 1 h to give an off-white suspension (pH˜5.5). Add 135 g of water into pot at 20° C. over 15 min to obtain a off-white suspension. Slowly add 10 g of 2N hydrochloric acid aq. solution into pot and adjust the mixture to pH 1-2. Filter off the solids through a polypropylene filter cloth in a Büchner funnel then wash the flask and filter cake once with 80 g of water. Dry the wet at 60±3° C. (20 mbar) for 18 h to give 26.3 g of (2R)-2,3-dihydro-1-[[4-[[5-methyl-2-[4-(trifluoromethyl)phenyl]-4-oxazolyl]-methoxy]phenyl]sulfonyl]-1H-indole-2-carboxylic acid (C6).

### C6→C6′

Initially obtained C6 from the above procedure forms long needles with electrostatic properties. The following procedure describes the modification of crystal behaviour using polyvinylpyrrolidone (PVP) as an additive during recrystallization of C6, resulting in a product (C6′) without electrostatic properties.

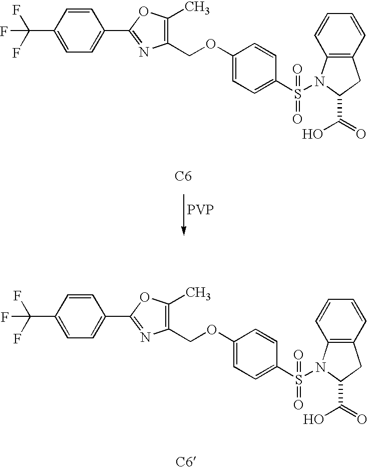

### Procedure:

Charge a 500-mL, 3-necked round bottom flask reactor (equipped with mechanical stirrer, nitrogen inlet, and thermocouple) with C6 (3.79 g, 6.7 mmol) and polyvinylpyrrolidone (PVP) (0.0380 g). Add 2-propanol (147.1 mL, 116.2 g), and water (4.6 mL, 4.6 g) and begin stirring the contents of the kettle. Heat the reactor contents to 74° C. over 0.25 h. Maintain the temperature at 74° C. for 0.5 h. Cool the reaction mixture to 20 3° C. over 4 h. Stir the resulting slurry at 20° C. for 8-12 h. Filter the solids from the slurry and rinse with 3% v/v water in 2-propanol (20 mL, 15.9 g). Allow the solids to dry for 1 h and then collect C6′. Dry the solids in a vacuum oven at 75° C./600 mm Hg below atmospheric pressure for 4-5 h, or until constant weight is achieved to give 3.38 g.

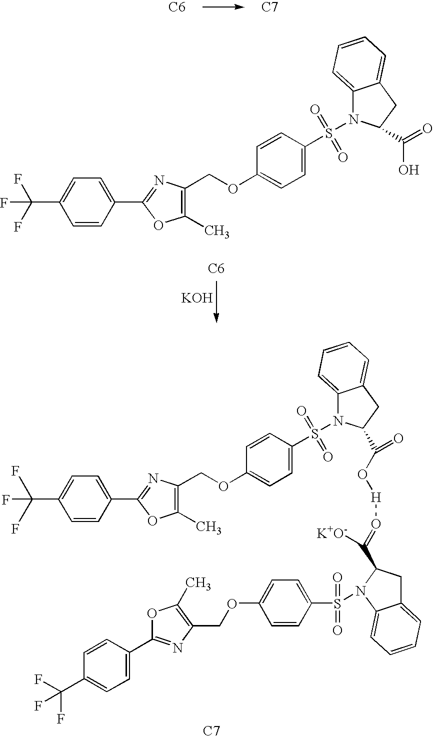

### Procedure:

Charge a 1-L reactor with 35.7 g (0.064 mol) of C6 and add 850 mL of isopropanol. Heat to an internal temperature of 70° C. Add a solution of 31.86 mL (0.032 mol) of 1 N potassium hydroxide and 38.8 g (38.8 mL) of water. Cool the reaction mixture to 62±3° C. and seed the reaction mixture with 10 mg of C7. Cool from 65° C. to an internal temperature 50° C. over 30 min. Hold at an internal temperature of 50° C. for at least 1 h. Cool from 50° C. to an internal temperature 0-5° C. over 1.5 h. Hold at an internal temperature of 0-5° C. Add 84.0 g (107 mL) of 2-propanol over at least 20 min and stir for at least 30 min at 0-5° C. Isolate the solid by filtration and wash the cake with 157.0 g (200 mL) of 2-propanol. Dry the cake on the funnel for 30 min, then dry in an oven at 50-55° C. (15 mbar) for at least 16 h or to yield a white solid, 27.7 g of C7. C4+C5→C7 (alternative procedure to C6→C7)

### Procedure:

Charge a 250-mL round-bottomed flask with 23.2 g (0.142 mol) of C5 140.0 g (140.0 mL) of water and add 12.5 g (0.156 mol) of 50% sodium hydroxide. Stir the homogeneous reaction mixture for 15 min at an internal temperature of 20-25° C. Add 168.91 g (190 mL) of tetrahydrofuran. Stir, for 5 min at an internal temperature of 20-25° C. Add 50.3 g (0.058 mol) of C4 in 5 equal portions over at least 25 min. Stir the resulting heterogeneous reaction mixture at 20-25° C. for at least 2 h. Add 432.9 g (480 mL) of ethyl acetate and stir the heterogeneous reaction mixture for 15 min. The resulting fine solids were removed by filtration and the cake was washed with 45.1 g (50 mL) of ethyl acetate. Wash the reaction mixture with 480 g (470 mL) of 5 wt % sodium bicarbonate. Stir the biphasic solution for 15 min at 20-25° C. Remove the lower aqueous layer and wash the top organic layer with 488 g (480 mL) of 1N hydrochloric acid. Stir the biphasic solution for 15 min at 20-25° C. Discard the lower aqueous layer.

Charge a 1-L reactor with 295.6 g of C6 free-acid solution and determine the amount of C6 free-acid present in the solution Concentrate the reaction mixture to a volume of 180-220 mL using a jacket temperature of 50 to 55° C. and 80 to 100 mm Hg vacuum. Add 392.5 g (500 mL) of 2-propanol, and concentrate the reaction mixture to a volume of 180-220 mL using a jacket temperature of 50 to 55° C. and 80 to 60 mm Hg vacuum. Remove a sample for solvent analysis. Add 628.0 g (800 mL) of 2-propanol, and concentrate to a final volume of 830 to 870 mL.

Heat to an internal temperature of 70° C. Add a solution of 31.86 mL (0.032 mol) of 1 N potassium hydroxide and 38.8 g (38.8 mL) of water. Cool the reaction mixture to 62±3° C. and seed the reaction mixture with 10 mg of C7. Cool from 65° C. to an internal temperature 50° C. over 30 min. Hold at an internal temperature of 50° C. for at least 1 h. Cool from 50° C. to an internal temperature 0-5° C. over 1.5 h. Hold at an internal temperature of 0-5° C. Add 84.0 g (107 mL) of 2-propanol over at least 20 min and stir for at least 30 min at 0-5° C. Isolate the solid by filtration and wash the cake with 157.0 g (200 mL) of 2-propanol. Dry the cake on the funnel for 30 min, then dry in an oven at 50-55° C. (15 mbar) for at least 16 h or to yield a white solid, 27.7 g of C7.

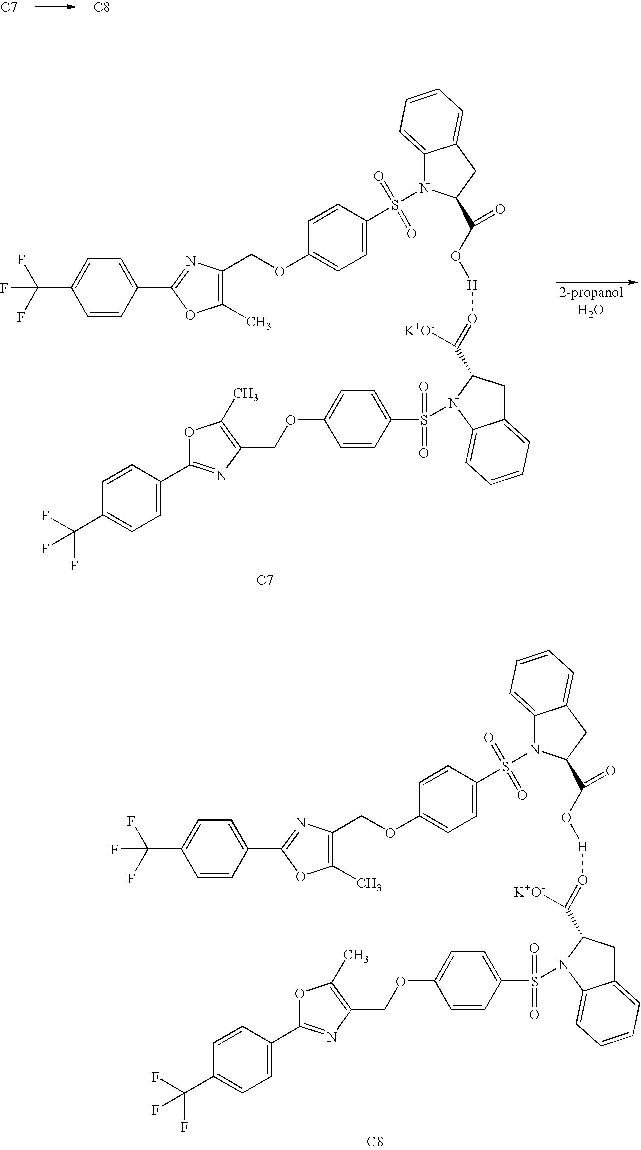

### Procedure:

Charge the 1-L reactor with 27.5 g (0.024 mol) of C7 and 720 g (917 mL) of 2-propanol Heat to an internal temperature of 70 to 75° C. Add 103 g (103 mL) of water. Cool to an internal temperature of 65° C. over 30 min and seed the reaction mixture with 10 mg of C7. Cool from 65° C. to an internal temperature 40° C. over 50 min. Hold at an internal temperature of 40° C. for at least 1 h. Cool from 40° C. to an internal temperature 0° C. over 80 min. Hold at an internal temperature of 0° C. for at least 1 h. Isolate the solid by filtration. Rinse the reactor with 63.2 g (81 mL) of 2-propanol into the Buechner funnel. Wash the cake with 157.0 g (200 mL) of 2-propanol. Dry the cake on the funnel for 30 min, then dry in an oven at 50-55° C. (15 mbar) for at least 16 h to yield a white solid, 24.9 g of C8.

What is claimed is:

1. A method of preparing a compound having the structure 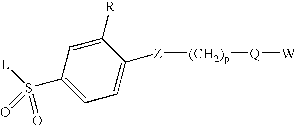
wherein L is 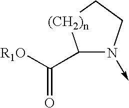
radical in which R₁ is hydrogen or optionally substituted alkyl, and n is zero or 1; or
L is 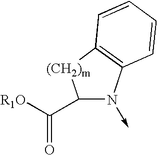
radical in which R₁ is hydrogen or optionally substituted alkyl, and m is 1;
wherein R is H, halogen, optionally substituted C₁₋₆alkyl or C₁₋₆alkoxy;
wherein Z is a bond, O or S;
wherein p is an integer from 1 to 5;
wherein Q is a bond provided that Z is not a bond when p is 1; or
Q is O, S or —C(O)NR₆— in which R₆ is hydrogen, optionally substituted alkyl or cycloalkyl; or
Q is —NR₆—, —NR₅C(O)NH— or —NR₅C(O)O— in which R₅ is hydrogen, alkyl or aralkyl provided that p is not 1;
wherein W is cycloalkyl, aryl or heterocyclyl; or
W and R₆ taken together with the nitrogen atom to which they are attached form a 9- to 10-membered bicyclic ring, which may be optionally substituted or may contain another heteroatom selected from oxygen, nitrogen and sulfur;
the method comprising the steps of
(a) reacting Cl—(CH₂)ₚ-Q-W in the presence of a solvent, and 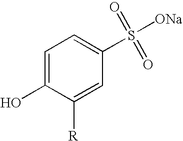
to provide 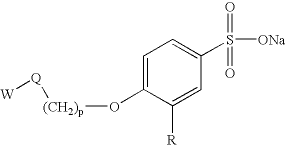
(b) treating the product of step (a) with dimethylformamide (DMF), acetonitrile, and thionyl chloride to provide 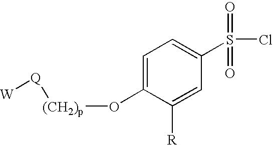
(c) reacting the product of step (b) with L in the presence of NaOH to provide the compound having the structure 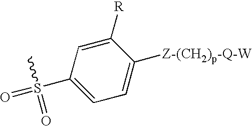
or a pharmaceutically acceptable salt thereof.

2. The method of claim 1, further comprising recrystallizing the product of step (c) in the presence of polyvinylpyrrolidone (PVP).

3. The method of claim 1,
wherein L is 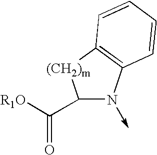
radical in which R₁ is hydrogen or optionally substituted alkyl, and n is zero or 1,
resulting in the compound having the structure

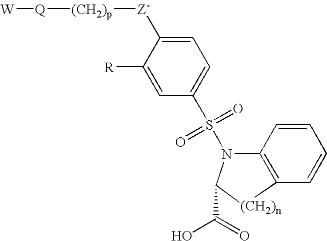

4. The method of claim 3, further comprising recrystallizing the product of step (c) in the presence of polyvinylpyrrolidone (PVP).

5. The method of claim 3, further comprising:
(d) treating the product of step (c) with KOH to provide the compound having the structure 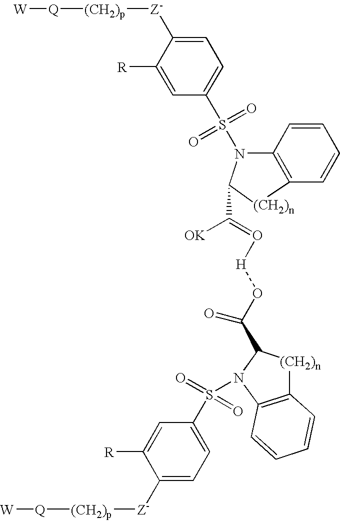
or having the structure 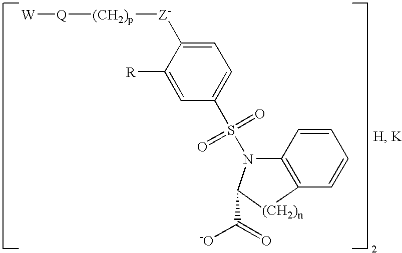
or having the structure

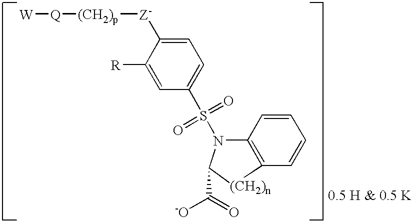

6. The method of claim 5 further comprising:
(e) recrystallizing the product of step (d).

7. The method of claim 6, wherein the recrystallizing is in the presence of a water miscible solvent and water.

8. The method of claim 7, wherein the water miscible solvent is 2-propanol.

9. The method of claim 6 further comprising
(f) milling the recrystallized product of step (e).

10. The method of claim 1,
wherein L is 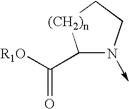
radical in which R₁ is hydrogen or optionally substituted alkyl, and m is 1,
resulting in the compound having the structure

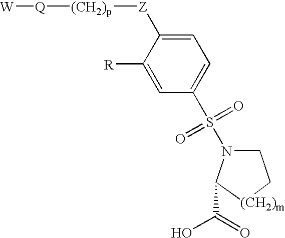

11. The method of claim 9, further comprising recrystallizing the product of step (c) in the presence of polyvinylpyrrolidone (PVP).

12. The method of claim 5, wherein R is a hydrogen, Z is O, p is 1, Q is a bond, and W is 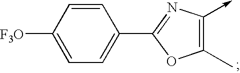
providing the product of step (c) followed by salt formation having the structure 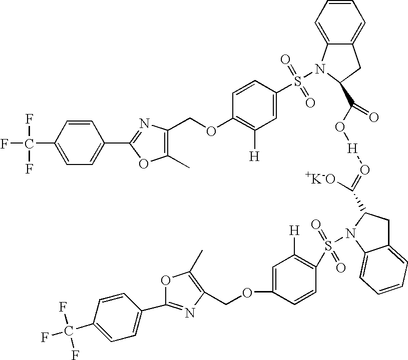
or having the structure 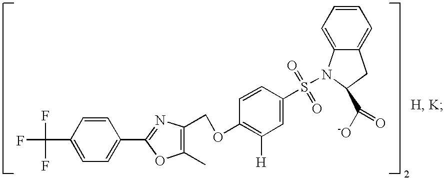
or having the structure 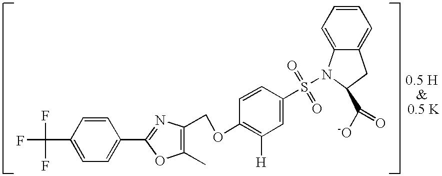
or an analogous pharmaceutically acceptable salt thereof.

13. The method of claim 12, wherein the analogous pharmaceutically acceptable salt is a magnesium salt or a calcium salt.

14. The method of claim 12 further comprising
(e) recrystallizing the product of step (d).

15. The method of claim 14, wherein the recrystallizing is in the presence of a water miscible solvent and water.

16. The method of claim 15, wherein the water miscible solvent is 2-propanol.

17. The method of claim 14 further comprising
(f) milling the recrystallized product of step (e).

18. The method of claim 1, wherein the solvent of step (a) is 1-methyl-2-pyrrolidinone (NMP), dimethylformamide (DMF), or dimethylacetamide (DMA).

19. The method of claim 1, wherein the product of step (b) is isolated by water quenching.

20. The method of claim 1, wherein R is H, halogen, optionally substituted C₁₋₆alkyl or C₁₋₆alkoxy; Z is O or S; p is 2; W is aryl or heterocyclyl; and Q is a —NR₆— in which R₆ is lower alkyl.

21. The method of claim 20, wherein R is H, chloro, n-propyl, or methoxy.

22. The method of claim 1, wherein R is H, halogen, optionally substituted C₁₋₆alkyl or C₁₋₆alkoxy, Z is a bond; p is 2; W is aryl or heterocyclyl, or W and R₆ taken together with the nitrogen atom to which they are attached form a 9- to 10-membered bicyclic ring, which may be optionally substituted or may contain another heteroatom selected from oxygen, nitrogen and sulfur; and Q is a —C(O)NR₆— in which R₆ is optionally substituted alkyl.

23. The method of claim 22, wherein R is H, chloro, n-propyl, or methoxy.

24. The method of claim 1, wherein R is H, halogen, optionally substituted C₁₋₆alkyl or C₁₋₆alkoxy; Z is a bond, O, or S; p is 2 or 3; W is aryl or heterocyclyl; and Q is O or S.

25. The method of claim 24, wherein R is H, chloro, n-propyl, or methoxy.

26. The method of claim 1, wherein R is H, halogen, optionally substituted C₁₋₆alkyl or C₁₋₆alkoxy; Z is a bond, O, or S; p is 2 or 3; W is selected from the group consisting of: 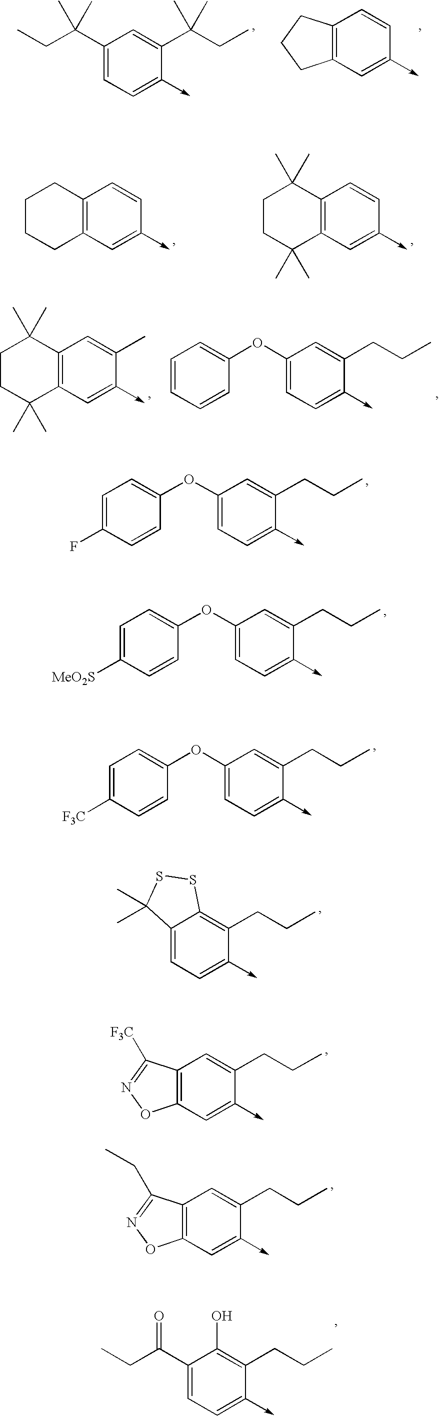 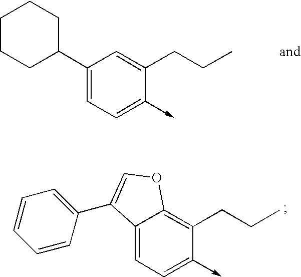
and Q is O or S.

27. The method of claim 1, wherein R is H, halogen, optionally substituted C₁₋₆alkyl or C₁₋₆alkoxy; Z is O or S; p is 1 or 2; W is aryl or heterocyclyl; and Q is a bond.

28. The method of claim 27, wherein R is H, chloro, n-propyl, or methoxy.

29. The method of claim 1, wherein R is H, halogen, optionally substituted C₁₋₆alkyl or C₁₋₆alkoxy; Z is O or S; p is 1 or 2; W is selected from the group consisting of: 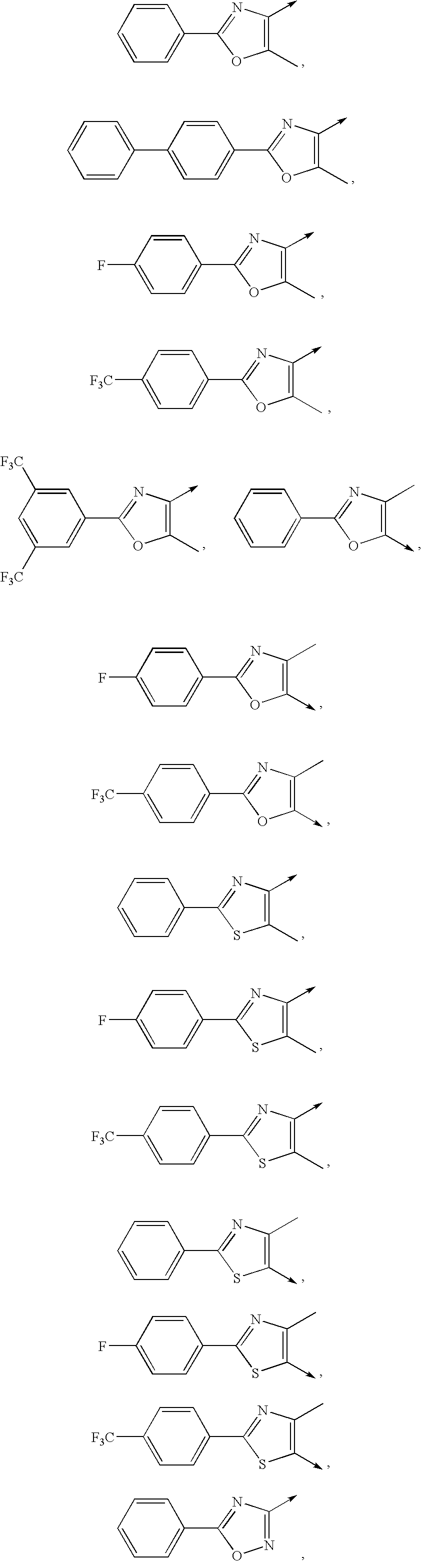 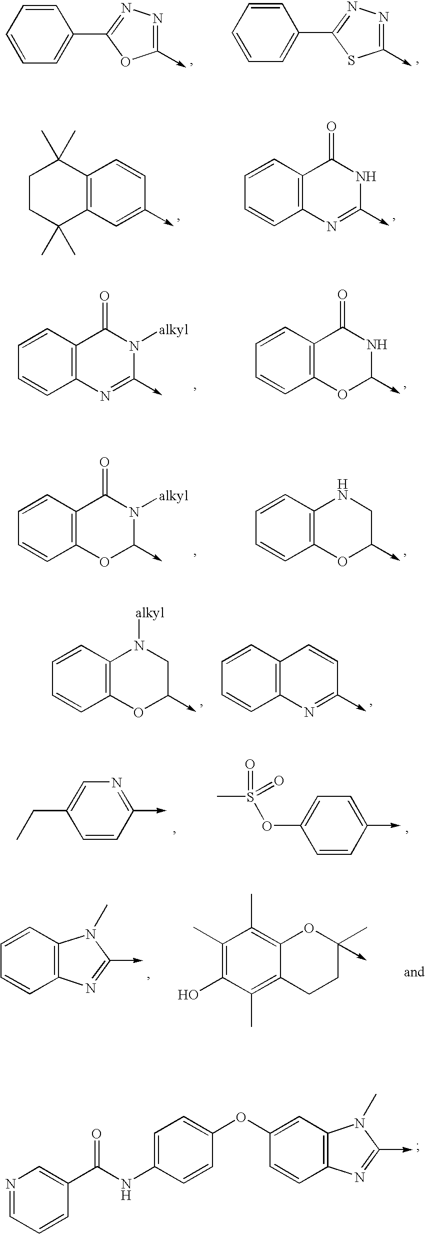
and Q is a bond.

30. The method of claim 1, wherein R is H, halogen, optionally substituted C₁₋₆alkyl or C₁₋₆alkoxy; Z is O or S; p is 2; W is selected from the group consisting of: 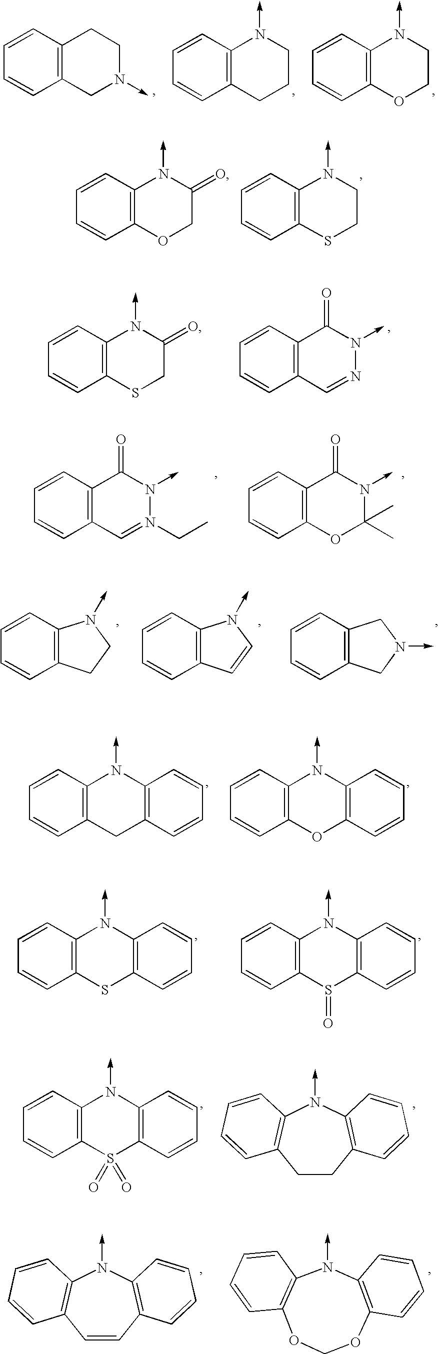 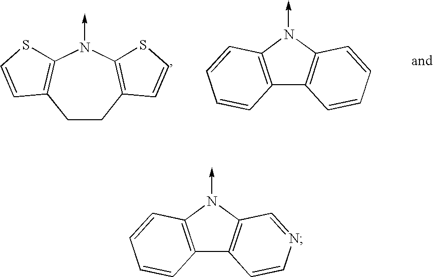
and Q is a bond.

31. A compound having the structure

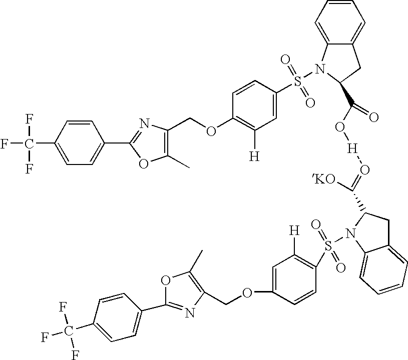

32. A compound having the structure

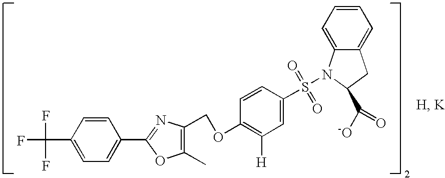

33. A compound having the structure [chemical structure]
